(19) United States
(12) Patent Application Publication
(10) Pub. No.: US 2010/0121239 A1
(43) Pub. Date: May 13, 2010

(54) SUPPORT INCLUDING STABILIZING BRACE AND INSERTS FOR USE WITH ANY NUMBER OF SPINAL VERTEBRAE SUCH AS UPPER THORACIC VERTEBRAE

(75) Inventor: Miguel A. Linares, BLOOMFIELD HILLS, MI (US)

Correspondence Address: GIFFORD, KRASS, SPRINKLE,ANDERSON & CITKOWSKI, P.C, PO BOX 7021, TROY, MI, 48007-7021, US

(73) Assignee: LINARES MEDICAL DEVICES, LLC, Auburn Hills, MI (US)

(21) Appl. No.: 12/614,472

(22) Filed: Nov. 9, 2009

Related U.S. Application Data
(60) Provisional application No. 61/112,959, filed on Nov. 10, 2008.

(51) Int. Cl. A61F 5/00

(52) U.S. Cl. 602/19

(57) ABSTRACT

A brace for securing any number of thoracic vertebrae with a composite plastic body exhibited an elongated stem. A plurality of clip engaging portions are formed in integrally extending fashion from the stem. Each of the clip portions terminate in an enlarged end portion which seats within an undercut location formed at specified locations within each of any sub-plurality of the vertebrae, and which are desired to be interconnected by the brace to prevent undesirable inter-movement of misaligned vertebrae.

### CROSS REFERENCE TO RELATED APPLICATIONS

The present application claims the benefit of U.S. Provisional Patent Application Ser. No. 61/112,959 filed Nov. 10, 2008.

### FIELD OF THE INVENTION

The present invention discloses a stabilizing/immobilizing brace for securing any number of thoracic vertebrae. Specifically, the brace prevents undesirable inter-movement of misaligned vertebrae (such resulting in great pain in instances where the spinal nerve is involved) and without the necessity of permanently and fixedly fusing the vertebrae in place. Additional features, such as inter-vertebral supporting inserts, can be incorporated in combination with or separately from the stabilizing brace and which provide additional biasing support at such as the inferior articular process locations where succeeding vertebrae normally contact each other.

### DESCRIPTION OF THE BACKGROUND ART

The prior art is documented with examples of spinal support plates and the like. In a most common example, a titanium plate is utilized and which his attached, such as by bone screws, to succeeding vertebral locations in order to fuse, or immobilize, a given area of the spine. Shortcomings associated with the installation of such fixed plates include both the pain and discomfort of implantation, along with the subsequent loss of flexibility.

### SUMMARY OF THE INVENTION

The present invention discloses a brace for securing any number of spinal vertebrae, and in particular a subset region of thoracic vertebra. The brace is constructed of a material including either a metal and/or plastic body (this further including a composite plastic) exhibited an elongated stem. A plurality of clip engaging portions are formed in integrally extending fashion from the stem. Each of the clip portions terminate in an enlarged end portion which resistively seats within an undercut location formed at specified locations within each of any sub-plurality of the vertebrae, and which are desired to be interconnected by the brace to prevent undesirable inter-movement of misaligned vertebrae

Each of the clip engaging portions may further incorporate first and second varying durometer rated portions, with the stem further exhibiting a third hardest durometer rated portion. Other features include biasing portions extending from the stem in order to bias against a vertebral location, such as in order to counter a scoliosis condition resulting from misaligned vertebrae. A key access hole can also be incorporated to allow for the angular repositioning of either or both of an integrally formed vertebral engaging clip portion and/or a separately extending biasing portion.

One or more multi-durometer rated plastic inserts can also be positioned for securing at both upper and lower opposing locations, this further including seating pockets defined between transverse processes associated with succeeding vertebrae. The inserts exhibit a three dimensional shape incorporating a fluid filled interior bladder and may also include mounting teeth or other irregularities for promoting bone growth following manipulated installation between selected vertebra, this including securing to opposite surfaces of a given transverse process to which the insert is mounted.

### BRIEF DESCRIPTION OF THE DRAWINGS

Reference will be made to the attached drawings, when read in combination with the following detailed description, wherein like reference numerals refer to like parts throughout the several views, and in which:

FIG. 1 is a plan view of a plurality of thoracic vertebrae and illustrating a pair of multi-durometer rated and elongated braces secured in lineal extending fashion;

FIG. 2 is an illustration of the pair of vertebral engaging braces in FIG. 1 and showing the configuration of the integrally formed clip portions for mounting the braces to undercut locations defined in each transverse extending process associated with a selected vertebrae;

FIG. 3 is a cutaway plan view taken along line 3-3 of FIG. 1 and illustrating the manner in which each of the clip portions associated with the first and second braces secure to their associated transverse process undercut locations;

FIG. 4 is an illustration of a pair of modified braces in which additional biasing portions are incorporated into the brace geometry, such as in order to counter a scoliosis condition resulting from misaligned vertebrae;

FIG. 5 is a cutaway top plan view illustration, similar to FIG. 3, and illustrating a pair of braces such as shown in FIG. 4 applied in a vertebral environment in which the biasing portions contact the downwardly/rearwardly angled shin process of an associated thoracic vertebrae;

FIG. 6 is a cross sectional illustration of a modified brace and showing the provision of a key access means which allows for the angular repositioning of the extending biasing leg;

FIG. 7 is a side plan illustration of the known prior art and referencing the positioning of the twelve thoracic vertebrae, collectively vertebral range 7, in relation to the various upper curve cervical vertebrae 1, 2, 3 and the lower positioned lumbar vertebrae 5, sacrum 6 and coccygeal vertebrae 4;

FIG. 7A is a further illustration according to the prior art and which illustrates the various features of a selected thoracic vertebrae;

FIG. 8 is an illustration of a subset series of thoracic vertebrae and further showing the provision of a multi-durometer rated plastic inserts for securing at both upper and lower opposing demi-facet locations established between succeeding body portions of each vertebrae;

FIG. 8A is a sectional illustration of a selected plastic supporting insert shown in FIG. 8;

FIG. 9 is a perspective illustration of a plurality of thoracic vertebrae such as also represented in FIG. 8 and illustrating one example of a three dimensional configured insert for assembling over either of a selected inferior or superior articular processes and for providing biasing and cushioning support associated which an opposing process of a succeeding vertebrae;

FIG. 9A is a side view of a selected pair of thoracic vertebrae shown in FIG. 9;

FIG. 9B is a cross sectional cutaway taken from FIG. 9A and illustrating a pair of inserts supported upon opposite inferior articular processes of a selected vertebrae in order to cushion against generally inner configured surfaces associated with the successive vertebrae;

FIG. 10 is a sectional perspective of the three dimensional insert shown in FIG. 9;

FIG. 10A is a further perspective of a slightly modified insert with a plurality of exteriorly facing teeth;

FIG. 10B is a slightly modified perspective compared to that shown in FIG. 10A and by which a pair of spaced teeth are substituted for the plurality of teeth;

FIG. 10C is a further modified perspective in which pairs of opposing and inward facing teeth are substituted to improve mounting to a selected inferior articular process; and

FIG. 10D is an illustration of an undercut mounting clip portion extending from the three dimensional insert of FIG. 10.

### DETAILED DESCRIPTION OF THE PREFERRED EMBODIMENTS

Referring now to the several illustrations, the present invention discloses a stabilizing brace for securing any number of thoracic vertebrae. As will be described throughout the succeeding embodiments, the brace is intended to provide any desired range of flexibility in application (as well as establishing substantial stationary immobilizing effect on the vertebrae in which it is installed).

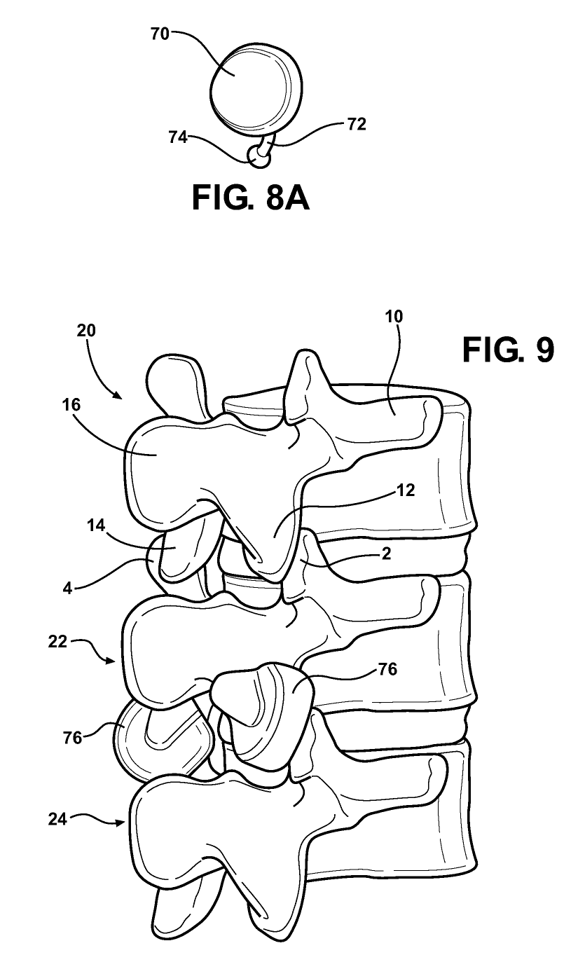

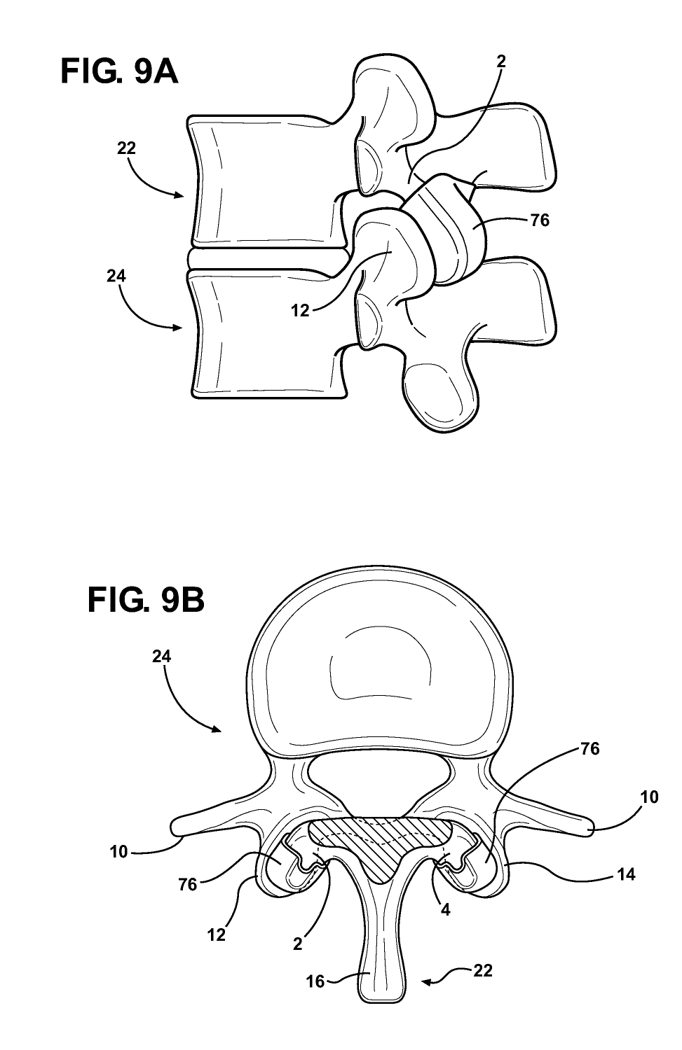

As will be further described, the brace can be provided individually or in a paired fashion and in order to prevent a given range of undesirable inter-movement of misaligned vertebrae, such movement being beyond a lesser range which may be desired and such as which may result in great pain in instances where impacting of the spinal nerve (not shown) and associated branches is involved, this without the necessity of permanently and fixedly fusing the vertebrae in place. As will be described in reference FIGS. 8 and 9, additional features such as inter-vertebral supporting inserts can be incorporated in combination with the stabilizing brace and which provide additional biasing support at such at the interface between opposing superior and inferior articular processes associated with succeeding vertebrae at the regions in which they normally contact each other.

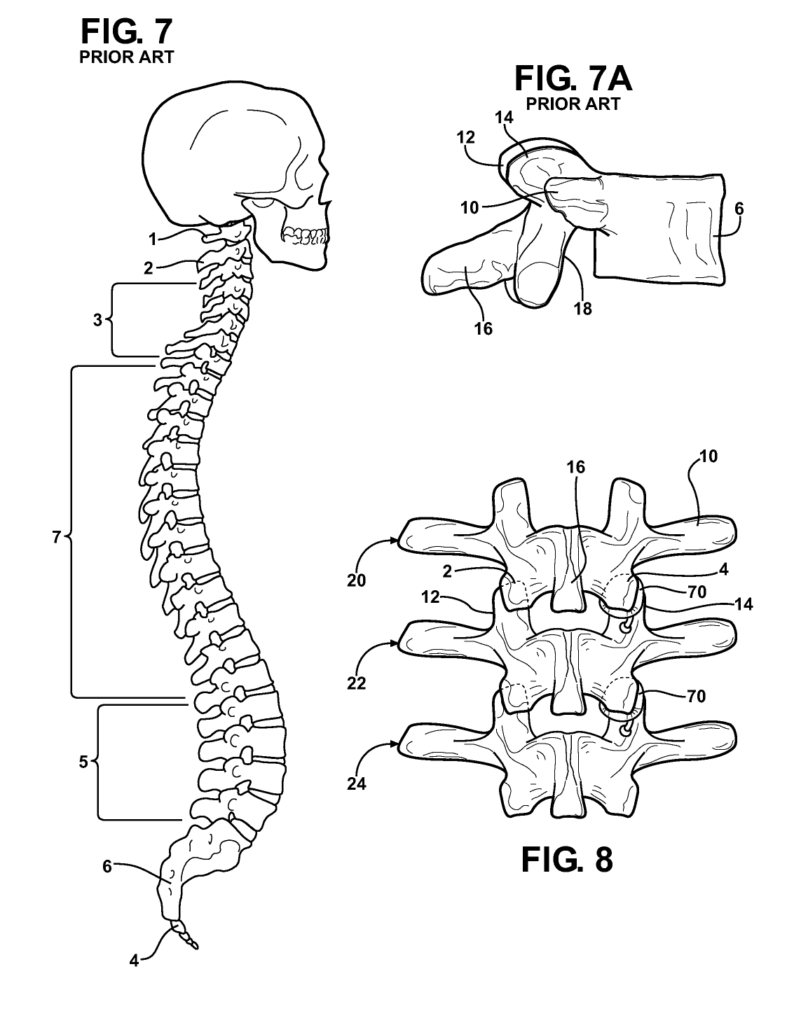

Referring first to FIG. 7, a side plan illustration is shown of the known prior art and referencing the positioning of the twelve thoracic vertebrae, collectively vertebral range 7, in relation to the various upper curve cervical vertebrae 1, 2, 3 and the lower positioned lumbar vertebrae 5, sacrum 6 and coccygeal vertebrae 4. The twelve thoracic vertebrae compose the middle segment of the vertebral column, between the cervical vertebrae and the lumbar vertebra.

The thoracic vertebrae are intermediate in size between those of the cervical and lumbar regions, with increasing size proceeding down the spine, the upper vertebrae being much smaller than those in the lower part of the region. As further shown in the prior art illustration of FIG. 7A, selected thoracic vertebrae is further distinguished by the presence of demi-facets, not shown but which are understood to be positioned on the sides of the main vertebral body 6 and which are positioned along opposite upper and downward surfaces of the selected vertebral body portion and which are further understood from an anatomical standpoint to progressively relocate from the top and bottom to the sides of the vertebral body, in particular from the 9^{th} through the 12^{th} thoracic vertebrae.

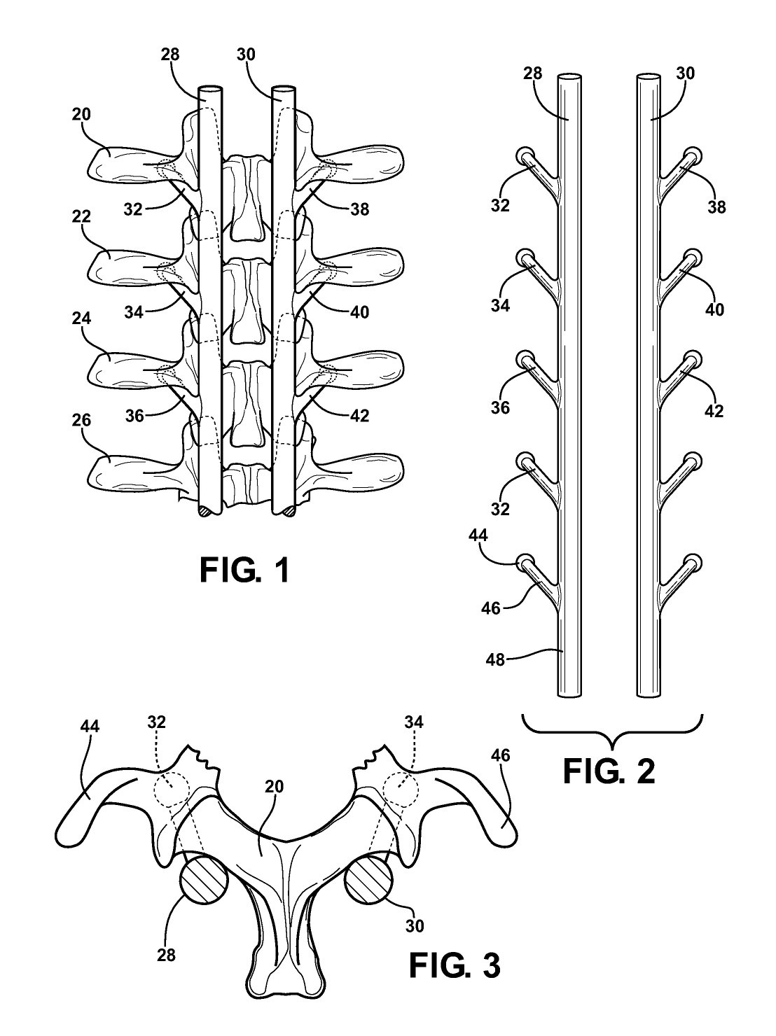

As will be disclosed in additional detail the recessed configuration of the upper and lower facets (these also being positioned on both of first and second lateral sides as shown in FIGS. 1 and 8) provide for articulation between succeeding vertebrae. Additional features associated with each thoracic vertebrae (and with the exception of the lowermost positioned eleventh and twelfth vertebrae) include transverse processes one of which is evident at 10 in FIG. 7A, upper and associated superior articular processes 12 and 14, a single downwardly and rearwardly extending shin process 16, and a pair of inner and inferior articular processes, see at 18. The inferior articular processes (these best illustrated at 2 and 4 in the cross sectional illustration of FIG. 9B) are fused to a considerable extent with the laminae, and project but slightly beyond their lower borders; their facets are directed forward and a little medialward and downward. The transverse processes arise from the arch behind the superior articular processes and associated pedicles, and are directed obliquely backwards and laterally, with each terminating in a clubbed extremity.

Referring now to FIG. 1 is a plan view is shown of a plurality of thoracic vertebrae, see as further represented at 20, 22, 24, 26, et. seq, and each of which again including the configuration of elements as depicted previously in the prior art illustration of FIG. 7A, as reinforced by the cross sectional view of FIG. 9B. Also illustrated are a pair of multi-durometer rated and elongated braces 28 and 30 secured in lineal extending fashion to each of a selected sub-plurality of the thoracic vertebrae. The braces and insert components according to the several preferred embodiments will be disclosed in use with mounting location particular to the thoracic vertebrae, with the further understanding that these can be redesigned or modified to the extent necessary to permit installation with such as the lumbar or even cervical vertebrae.

As also shown in FIG. 2, the pair of vertebral engaging braces 28 and 30 employed in FIG. 1 are constructed of a composite and, typically sanitary and durable plastic material exhibiting any necessary or desired degree of rigidity and flexibility. The braces 28 and 30 each include an elongate stem, from which extend (at any desired angle) integrally formed clip portions (also termed legs) such as which are shown at 32, 34, 36, et. seq. for brace 28 and further at 38, 40, 42, et. seq., for brace 30. Without limitation, the present invention contemplates any plastic or metal material, composite or admixture employed in the creation of the braces or, in further reference to FIGS. 8 et seq., the various inserts forming a part of the present invention.

As further shown in FIG. 2, indicated locations 44, 46 and 48 correspond to varied durometer (hardness) rated locations associated with the selected brace 28, with location 44 defining a softest durometer rated portion at an enlarged head or tip location of a selected clip portion (this engaging within a machined undercut location of a selected vertebrae as shown in FIG. 1). An intermediate and connecting location 46 between the enlarged head 44 and the stem corresponds to a likewise intermediate durometer rated portion of the selected vertebral clip. Location 48 further corresponds to a highest durometer rated portion, this corresponding to the stem of the indicated brace 28.

As again illustrated in FIG. 1, when viewed in combination with the top plan view of FIG. 3, the elongated stems 28 and 30 (following being sectioned to an appropriate length for position-ally interconnecting a selected sub-plurality of thoracic vertebrae) are aligned in generally lengthwise extending fashion with each of the laterally projecting transverse processes associated with the successively positioned vertebrae. It is understood that the elongated braces 28 and 30 can be utilized either singularly or in combination in the manner shown, and so that the braces immobilize the desired number of vertebrae in a similar manner to prior art medical procedures such as fusing, this in order to reduce pain such as resulting from misalignments between the vertebrae and which can further cause pinching of the associated spinal column and branching nerves (not shown).

As further shown in cutaway view of FIG. 3, the elongated braces 28 and 30 are shown with uppermost selected and angularly disposed clip portions 32 and 34, respectively, mounting the braces to undercut locations defined in each transverse extending process (further illustrated in this view at 44 and 46) associated with selected vertebrae 20. As is known, the undercut locations formed in each of the vertebrae can be created by employing a known bone drill with orbital bit, the configuration of the resultant undercut recess firmly engaging the brace upon its selected (and most softest plasticized enlarged clip end) being biasingly inserted within the undercut space. In this fashion, the clip portions associated with each brace are secured within similar undercut locations formed in each of the transverse processes of each of the succeeding vertebrae.

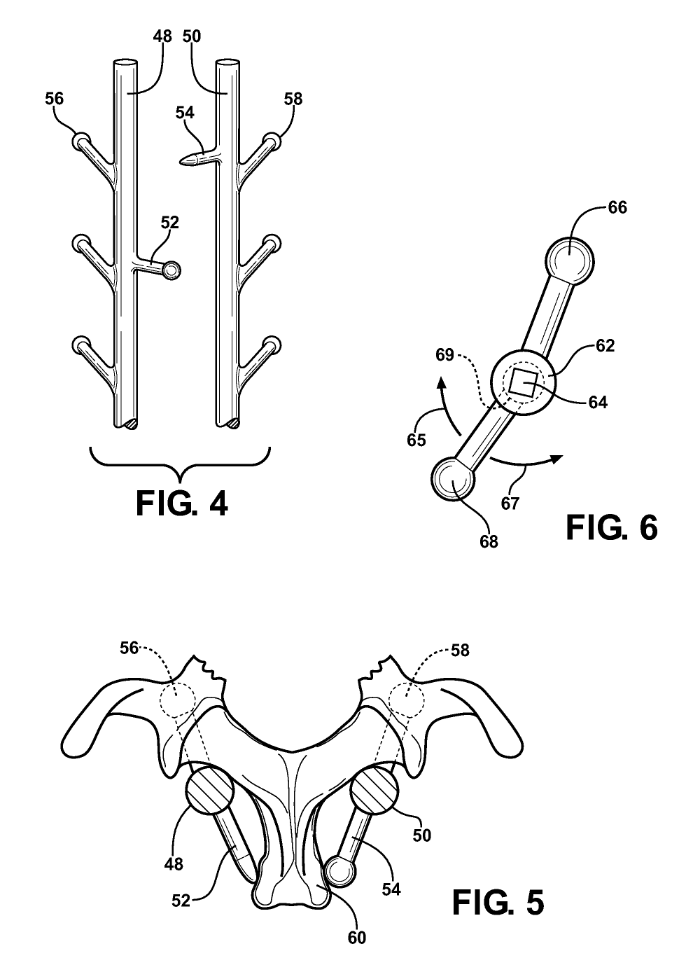

Proceeding to FIG. 4, an illustration is shown at 48 and 50 of a pair of modified composite plastic braces, these being constructed as substantially shown in each of FIGS. 1-3 with plural angular and integral extending engagement clip portions. Each of the redesigned braces 48 and 50 additionally include biasing portions, see selected biasing portions at 52 and 54, which are incorporated into the brace geometry (and as shown extend in another direction in integral fashion from the stem of the associated brace such as at 48), such as in order to counter a scoliosis condition resulting from misaligned vertebrae. This condition is illustrated in the plan cutaway view of FIG. 5 and which illustrates the pair of braces shown in FIG. 4 applied in a vertebral environment (with selected clips 56 and 58 engaging the vertebral transverse processes in likewise undercut machined and resistive fitting manner), and further in which biasing portions 52 and 54 are positioned so that they contact the downwardly/rearwardly angled (and misaligned) shin process 60 of an associated thoracic vertebrae. In this fashion, the modified braces 48 and 50, in addition to stabilizing/immobilizing a selected plurality of vertebrae, provide the additional benefit of addressing (such as by either stabilizing or straightening) a scoliosis condition associated with any sub-plurality of the vertebrae.

Further illustrated in FIG. 6 at 62 is a cross sectional (2D) illustration of a modified brace and showing the provision of a key access means, see key hole 64. A matingly configured bit inserting portion, such associated with such as a screwdriver (not shown), upon being inserted within the key hole 64, allows for the angular repositioning (see directional arrows 65 and 67) of either or both of an integrally formed vertebral engaging clip portion 66 and/or a separately extending biasing leg 68. This is accomplished via any number of rotational adjustable structure incorporated between the stem location (again at 62) of the brace and the respective biasing legs or clips 66 and 68. In on non-limiting possible variant, and although not clearly illustrated, it is envisioned that the inner mounting ends of the legs/clips 66 and 68, as further referenced in phantom at 69 as to clip 68 in FIG. 6, can be mounted within an angular slot or track defined in the stem 62 and such that rotation of the inserting bit results in a limited degree of angular readjustability of the associated clip 68.

Referring now to FIG. 8, an illustration is provided of a subset series of thoracic vertebrae, see again at 20, 22 and 24, and which are supported by the provision of one or more multi-durometer rated plastic inserts 70 (also shown in FIG. 8A) positioned for securing at both upper and lower opposing locations established between succeeding body portions of each vertebrae. As further shown in FIG. 8A, the selected plastic supporting insert 70 exhibits a generally rounded and three dimensional shaped body (such as a generally combination pseudo disk and cushion configuration which can include any of a crescent, arcuate or other 3D profile), and which further includes an integrally formed and extending clip (or angled leg) portion 72 terminating in an enlarged tip portion 74 (the insert 70, clip 72 and enlarged tip 74 again including such as harder to progressively softer durometer rated composite plastics as previously described). The configuration of the supporting insert 70 is such that it can optionally include a biasing (i.e. fluid filled) interior bladder such that, when placed in a generally recessed pocket shaped area identified by opposing superior and inferior articular process locations, provides additional biasing and cushioning support.

FIG. 9 is a perspective illustration of a plurality of thoracic vertebrae such as also represented in FIG. 8 and illustrating one example of a three dimensional configured insert, see pair of inserts shown at 76 for assembling over either of a selected inferior articular process (as shown in particular in FIG. 9B in reference to inferior articular processes 2 and 4). Although not illustrated, it is also understood that the insert 76 can also be reconfigured for mounting over the opposing associated superior articular processes 12 and 14 for providing biasing and cushioning support associated which an opposing process of a succeeding vertebrae.

FIG. 9A is a side view of a selected pair of thoracic vertebrae 22 and 24 shown in FIG. 9. In combination with FIG. 9B again further illustrating a cross sectional cutaway illustrating the pair of inserts 76 supported upon opposite inferior articular processes 2 and 4 of selected vertebrae 22, the inserts 76 are configured so that, upon assembly such as over the inferior articular processes, provide cushioning support at the lateral seating locations established between the inferior and superior articular processes.

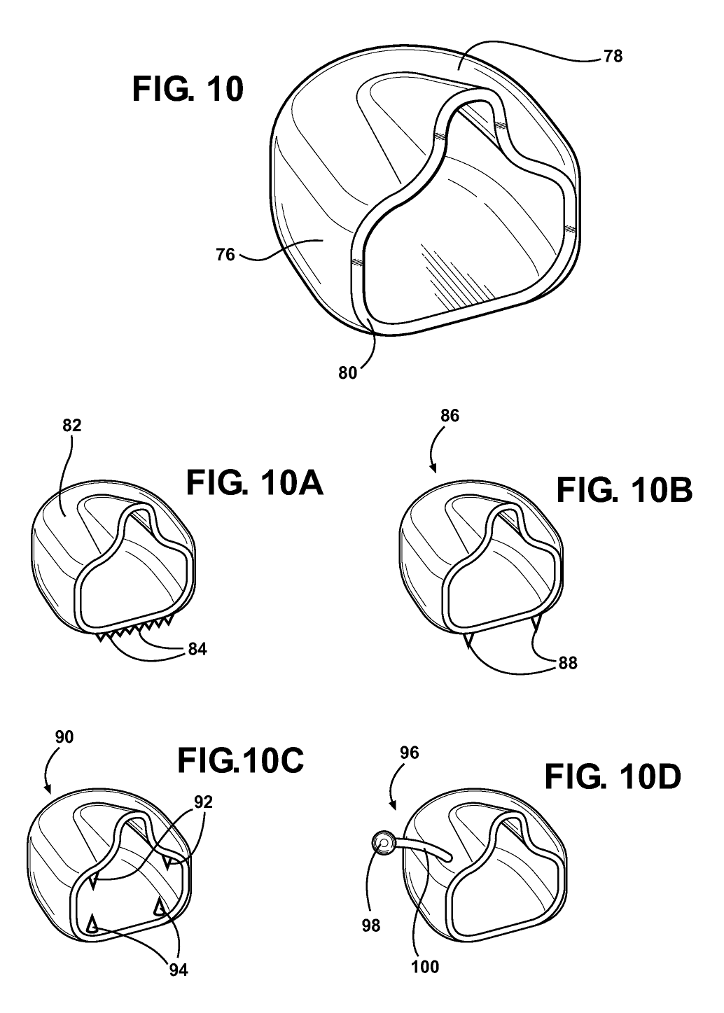

Referring now to FIG. 10, a sectional perspective is illustrated in enlarged fashion of the three dimensional shaped insert 76 shown in FIG. 9. Specifically, the insert 76 exhibits a generally arcuate and interiorly hollowed shape and, as previously described, can be constructed of any suitable material (such as a varying durometer plastic but also contemplating a lightweight and ductile metal) exhibiting sufficient flexibility for installation over the extending end portion of the associated spinal process (e.g. inferior articular process as shown).

As further shown in FIG. 10, the pocket defining configuration of the insert 76 includes an edge defined projecting location 78 associated with the overall rim opening 80. In operation, the configuration, including shape and thickness, of the insert 76 can include any of an arcuate, dome, crescent or like shape which facilitates proper fit over the associated spinal process location and in order to establish a cushioning surface at a selected vertebral interface, this further such as to compensate for any misalignment and/or wear associated with the given seating pocket defined between the opposing pair of superior and inferior articular vertebral processes.

FIG. 10A is a further perspective of a slightly modified insert 82 with a plurality of exteriorly facing teeth 84 established along a bottom surface and which facilitates either or both of frictional location as well as bone inducing growth in order to anchor the insert to the associated spinal process. FIG. 10B is a slightly modified perspective illustration 86 of an insert, as compared to that shown in FIG. 10A and by which a pair of spaced teeth 88 are substituted for the plurality of teeth.

FIG. 10C is a further modified perspective view of an insert 90 in which pairs 92 and 94 of opposing and inward facing teeth are substituted to improve mounting to a selected inferior articular process. Finally, FIG. 10D is an illustration of a further example 96 of a three dimensional insert and in which an undercut mounting clip portion including an enlarged head 98 which extends from an interconnecting leg 100 of the insert for undercut mounting to a given vertebral bone location and in a similar fashion as previously described in reference to the clip in FIG. 8A.

Having described my invention, other and additional preferred embodiments will become apparent to those skilled in the art to which it pertains, and without deviating from the scope of the appended claims.

I claim:

1. A brace for securing any number of thoracic vertebrae, comprising:
a composite plastic body exhibiting an elongated stem;
a plurality of clip engaging portions extending from said stem, each of said clip portions terminating in an enlarged end portion which seats within an undercut location formed within each of any sub-plurality of the vertebrae which are desired to be interconnected by said brace and which prevents undesirable inter-movement of misaligned vertebrae.

2. The brace as described in claim 1, each of said clip engaging portions further comprising first and second varying durometer rated portions.

3. The brace as described in claim 2, said stem further comprising a third hardest durometer rated portion.

4. The brace as described in claim 1, further comprising biasing portions extending from said stem in order to bias against an external vertebral location, such as in order to counter a scoliosis condition resulting from misaligned vertebrae.

5. The brace as described in claim 4, further comprising a key access hole allowing for the angular repositioning of either or both of an integrally formed vertebral engaging clip portion and/or a separately extending biasing portion.

6. The brace as described in claim 1, further comprising at least one three dimensional shaped plastic insert securing at an opposing interface established between succeeding vertebrae.

7. The brace as described in claim 6, said plastic supporting insert further comprising a rim defining an open interior.

8. The brace as described in claim 6, said insert further comprising a generally rounded or arcuate configuration establishing a fluid filled interior bladder.

9. The brace as described in claim 6, said insert further comprising at least one of teeth, spikes, or clip engaging portions.

10. The brace as described in claim 9, said insert further comprising undercut mounting teeth for securing between opposing surfaces of a pair of succeeding vertebral bodies.

11. The brace as described in claim 7, said insert rim further comprising an edge projecting location which is manipulated during installation over a selected articular process.

12. A support for aligning spinal vertebrae, comprising:
a plasticized body; and
at least one engaging portion associated with said body for securing to a selected vertebral bone location in order to position said body between an interface location established between succeeding vertebrae and which prevents undesirable inter-movement between the vertebrae.

13. The invention as described in claim 12, said body further comprising an elongated stem, said engaging portion further comprising a plurality of clip engaging portions integrally extend from said stem.

14. The invention as described in claim 13, further comprising each of said clip engaging portions establishing first and second varying durometer rated portions, said stem further comprising a third hardest durometer rated portion.

15. The invention as described in claim 13, further comprising an additional portion extending from said stem apart from said clip engaging portion and in order to bias against an external vertebral location.

16. The invention as described in claim 13, further comprising said clip portion being adjustable relative to said body via a key access hole allowing for the angular repositioning of said clip portion.

17. The brace as described in claim 12, said body further comprising a generally rounded or arcuate configuration establishing a fluid filled interior bladder.

18. The brace as described in claim 12, said body exhibiting a three dimensional shape including a rim defining an open interior and further comprising an edge projecting location which is manipulated during installation over a selected articular process.

19. The brace as described in claim 12, said engaging portion further comprising a clip engaging portion extending from said body and terminating in an enlarged end portion adapted to seat within an undercut location formed within a selected vertebrae.

20. A brace for securing any number of thoracic vertebrae and which prevents undesirable inter-movement of misaligned vertebrae, comprising:
a composite plastic body exhibiting an elongated stem;
a plurality of clip engaging portions extending from said stem, each of said clip portions terminating in an enlarged end portion which exhibit a softest durometer rating and which seats within an undercut location formed within each of any plurality of the vertebrae which are desired to be interconnected by said brace; and
connecting portions extending between said enlarged end portion and said stem each exhibiting an intermediated durometer rating, said stem further exhibiting a hardest durometer rating.
